(19) United States
(12) Patent Application Publication
(10) Pub. No.: US 2012/0024285 A1
(43) Pub. Date: Feb. 2, 2012

(54) LARYNGEAL MASK AIRWAY PLACEMENT SYSTEM AND METHOD

(76) Inventor: Roman W. Dashawetz, Westwood, MA (US)

(21) Appl. No.: 12/848,998

(22) Filed: Aug. 2, 2010

(51) Int. Cl. A61M 16/00

(52) U.S. Cl. 128/200.26

(57) ABSTRACT

A laryngeal mask airway having a laryngeal mask and a flexible airway tube is provided with an external brace that is easily detached or reattached onto the airway tube. The brace, when attached, enhances the stiffness of the combination to facilitate placement of the mask at the laryngeal inlet of a patient. After the mask is in proper, sealed position at the laryngeal inlet the brace may be removed to permit the external portion of the flexible airway tube to be located in an out-of-the-way position.

### FIELD OF THE INVENTION

The invention relates to laryngeal mask airways and methods and devices to facilitate their placement.

### BACKGROUND OF THE INVENTION

This invention relates to improvements in laryngeal mask airways to provide ventilation for an unconscious person or to administer general anesthesia to a patient undergoing surgery. Ideally, these procedures require the establishment of a sealed airflow path to the trachea that also prevents reflux of gastric contents from reaching the patient's airway while minimizing trauma to surrounding tissues. Use of laryngeal mask airways has developed as an alternative to face masks and endotracheal tubes.

Facemasks do not seal as effectively as may be desired and may tend to leak. Additionally they cannot be used when the surgery involves the nose, oral cavity and throat. While an endotracheal tube provides a more effective seal, it may present other difficulties. An endotracheal tube typically has an inflatable cuff adjacent its leading end. The tube is inserted through the mouth or nose and is advanced through the larynx and vocal cords into the trachea. When so positioned the cuff is inflated to press against the inner surface of the trachea to establish an effective airway seal as well as to prevent reflux of gastrointestinal fluids into the trachea from the esophagus. The insertion of an endotracheal tube and inflation of the cuff is a delicate procedure and presents risk of trauma to the delicate tissues and linings of the region about and in the oral cavity, the trachea, pharynx and the larynx. Notwithstanding these potential difficulties, endotracheal tubes are in wide use.

Another, less traumatic, technique for providing ventilation and administration of anesthesia during surgery has been the development of the laryngeal mask airway (“LMA”). The LMA incorporates a mask that can be advanced through the mouth and oropharynx and positioned to overlie the laryngeal inlet, just above the larynx. The mask includes an oval-shaped inflatable cuff adapted to seal about the laryngeal inlet to isolate the trachea from the esophagus, thus providing a direct airway to the trachea and lungs while preventing reflux of gastrointestinal fluids into the trachea while the mask is sealed in place. The LMA does not extend into the trachea, does not traverse the vocal cords and avoids the risk of injuring those tissues and organs as may result from use of an endotracheal tube.

Although the use of an LMA provides a number of advantages and avoids many of the risks and difficulties encountered with endotracheal tubes, in some cases positioning the mask to obtain a proper seal about the laryngeal inlet may present some difficulty. In those cases it is not uncommon for the anesthetist to have to reach deeply with his fingers into the oropharyngeal cavity in order to adjust the position of the mask against the tissue that defines the laryngeal inlet. That is common particularly with those LMAs in which the airway tube is highly flexible. Highly flexible airway tubes are often desirable where the surgery to be performed is in the region of the head, neck and oral cavity because the flexibility of the airway tube enables it to be bent out of the way without obstructing airflow and taped in position so as to minimize interference with the operation. While such flexible LMAs provide significant advantages in certain settings, they also may be difficult to place because the flexibility of the airway tube provides insufficient leverage to manipulate the mask within the patient and provides reduced tactile feedback to help determine if the mask has made a proper seal. Consequently, the practitioner frequently must manipulate the mask of a flexible LMA into place by reaching deeply into the oropharynx with the fingers.

Another consideration in the selection and placement of the ventilating and anesthesia delivery apparatus is the length of time that it may take to put the apparatus in place and operation. For some patients, e.g., those for whom oxygen saturation may become depleted rapidly, it is essential that the placement be completed as quickly as possible. When using an LMA, particularly one with a flexible airway tube, the flexibility of the airway tube may hinder placement and result in delay.

It would be desirable to provide a device and technique by which a flexible LMA can be placed more easily, quickly and effectively.

### SUMMARY OF THE INVENTION

In accordance with the invention an airway tube of a flexible LMA is temporarily stiffened by an airway brace that is detachably mounted on the airway tube. The brace is mounted to the airway tube during placement of the LMA and is removed after the LMA is deployed in the patient. The brace provides for better control in the placement of the mask. Once the mask has been positioned and an effective seal has been confirmed, the brace can be detached and the flexible airway tube can be flexed and taped in an out-of-the-way location for the duration of the surgical procedure.

The brace is mounted exteriorly and securely to the airway tube. The brace may take a number of forms. One embodiment may be in the form of a generally tubular member having a longitudinally extending, laterally expandable slot or slit by which the brace can be slipped onto the airway tube. The resulting combined airway tube and brace provides an assembly that is stiffer than the flexible airway alone and has sufficient stiffness to facilitate positioning the mask by manipulation of the stiffened airway tube. The braced device may be at least as stiff as a standard semi-rigid airway tube of corresponding size. The brace may be formed from any number of materials and configured to be readily detachable from the airway tube. By increasing, temporarily, the stiffness of the airway tube, the placement of the mask is more easily controlled and that enables a quicker, more accurate placement of the mask cuff. The stiffening brace may be provided with various pre-formed curves when in a relaxed state. In other embodiments the brace may have a manually shapeable member extending longitudinally of the brace to enable the practitioner to shape the curve of the LMA as desired to better conform to the anatomy of a particular patient. In other embodiments portions of the brace may be constructed to have increased flexibility along its leading portion.

### DESCRIPTION OF THE DRAWINGS

The advantages and features on the invention will be appreciated more fully from the following description and the accompanying drawings in which:

FIG. 1 is an illustration of the general configuration of an LMA device;

FIG. 2 is a longitudinal sectional illustration of the mask at the leading end of the LMA as seen along the line 2-2 of FIG. 1;

FIG. 3 is a diagrammatic illustration of an LMA positioned over and sealed against the laryngeal inlet within a patient;

FIG. 4 is a side view of an LMA with the a stiffening brace attached to the airway tube of the LMA;

FIG. 5 is an illustration of the brace as seen from its posterior side, showing the configuration of the slot;

FIG. 6 is a cross-sectional illustration an embodiment of the combined brace and LMA as seen along the line 6-6 of FIG. 4;

FIG. 7 is a cross-sectional illustration an embodiment of the combined brace and LMA as seen along the line 7-7 of FIG. 4;

FIG. 8 is a cross-sectional illustration an embodiment of the combined brace and LMA as seen along the line 8-8 of FIG. 4;

FIG. 9 is a cross-sectional illustration, similar to FIG. 7, showing a modified embodiment in which the brace has a manually shapeable stylet to enable the braced LMA to be adapted to conform to the anatomy of a patient;

FIG. 10 is a longitudinal cross sectional illustration of the trailing end of a brace having a shapeable stylet;

FIG. 11 is an illustration of the manner in which the brace is peeled away from the airway tube of the LMA;

FIGS. 12-15 illustrate embodiments of braces in which leading segments are modified to have greater flexibility than the trailing segments;

### DESCRIPTION OF THE ILLUSTRATIVE EMBODIMENTS

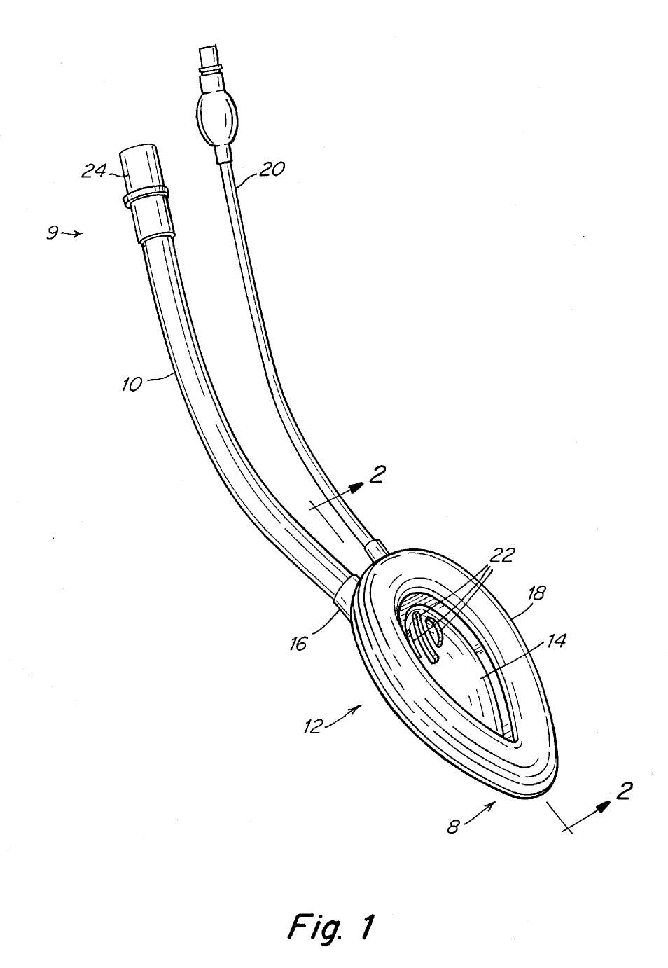

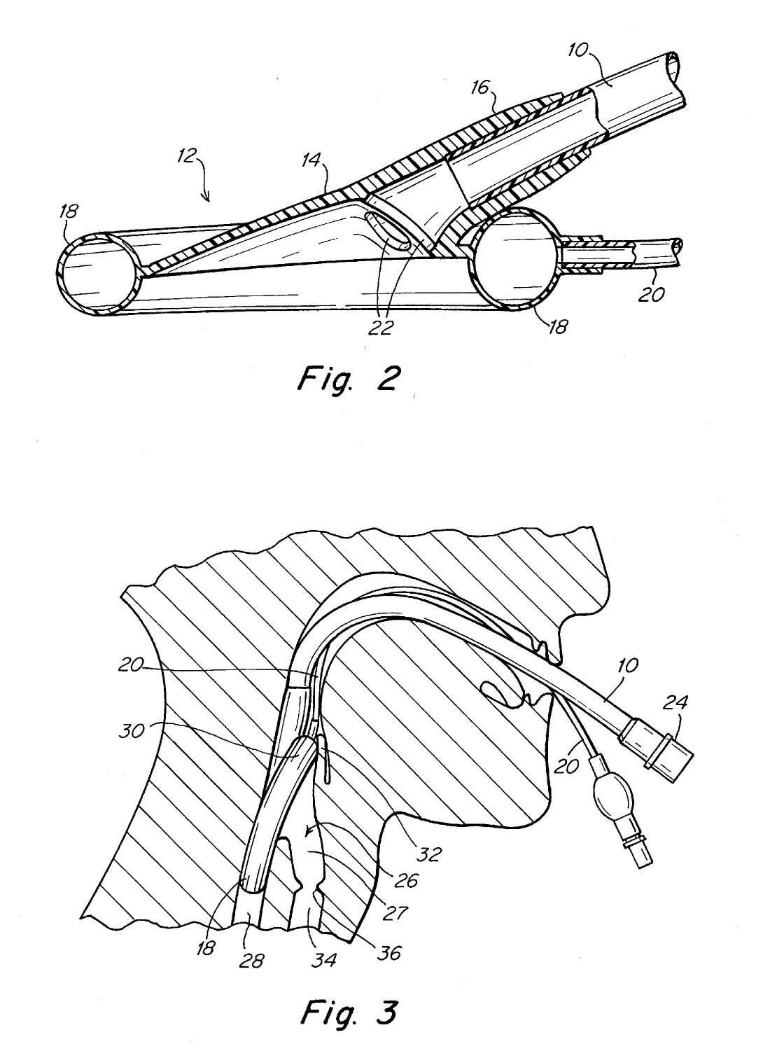

FIGS. 1 and 2 illustrate the general configuration of a laryngeal mask airway (LMA). The device may be considered as having leading and trailing ends 8, 9 and includes an airway tube 10 and a mask 12 attached to the leading end 8 of the tube 10. The direction toward the leading end 8 of the device may be referred to as “proximal” and the opposite direction toward the trailing end 9 may be referred to as “distal”.

The mask 12 includes a base 14 having a fitting 16, at which the airway tube 10 is attached, and an inflatable cuff 18 attached to and about the periphery of the base 14. The cuff 18 is inflatable through an inflation tube 20 that communicates with the interior of the cuff. The base 14 of the mask includes one or more apertures 22 by which the lumen of the airway tube 10 communicates with the interior of the mask. In use, the LMA is connected to a ventilator by a connector 24 at the trailing end of the airway tube 10 through which oxygen and anesthetic gases may be delivered to the patient. The oval-shaped cuff in 18 is shaped to fit over the laryngeal inlet, adjacent to the entry to the patient's trachea. After the mask has been positioned, the cuff 18 is inflated to create a seal about the laryngeal inlet to establish a sealed flow passage from the airway tube 10 through the apertures 22 to the trachea.

The components of the LMA typically are formed from polymers commonly used for such devices and are well known in the art. The cuff 18 typically is formed from a relatively soft, flexible polymer, such as silicone rubber. The base 14 and fitting 16 also may be formed from silicone rubber. Inflation tube 20 and airway tube 10 may be formed from stiffer polymeric materials such as PVC. LMAs are available commercially in a variety of sizes ranging from pediatric to adult.

A “standard” LMA typically has a semi-rigid airway tube 10 with a preformed gradual curve that, typically, must be further bent during placement to accommodate the anatomical curve of the patient's oropharynx. The semi-rigid airway tube has a degree of stiffness that enables the practitioner to position the airway mask by manipulating the airway tube. Once placed, however, the airway tube projects straight out of the patient's mouth and may interfere with surgical procedures in the region of the head. Additionally, the semi-rigid airway tube tends to bear firmly against the back of the throat and may cause trauma and result in post-operative soreness. In contrast, a “flexible” LMA has an airway tube that is substantially more flexible than the standard LMA so that it is better adapted to conform to the curve of the patient's oropharynx anatomy resulting in less risk of trauma and, after placement, the airway tube 10 can be flexed to and secured in an out-of-the-way position. A flexible LMA also can be expected to apply less pressure on the lips and tongue as compared to the standard LMA. Additionally, should it be necessary to reposition the patient during the surgery, use of a flexible LMA presents less risk of the LMA becoming dislodged. Although the airway tube of a flexible LMA typically has a helical coil embedded in its wall to prevent kinking or radial collapse, it does not have sufficient bending rigidity to enable the position of the mask to be adjusted by manipulating the airway tube, as with the standard LMA. It is the general object of the invention to enable the benefits of the flexible LMA to be realized while also enabling it to be placed with at least the same facility as a standard LMA.

FIG. 3 illustrates the manner in which the LMA is placed. The leading, proximal end of the device is inserted through the mouth and oropharynx to locate the mask at the region of the laryngeal inlet 26. The proximal, leading end of the mask may be placed at the upper end of the esophagus 28 and the heel portion 30 of the mask bears against the epiglottis 32. The shape of the mask, particularly the cuff 18, is designed to fit over and seal against the laryngeal inlet 26 leading to the trachea 34 when the device is properly placed. When in position the cuff 18 is inflated and forms a seal that isolates the larynx 27, vocal cords 36 and trachea from the esophagus 28 while providing a flow passage through the airway tube and mask to ventilate the patient.

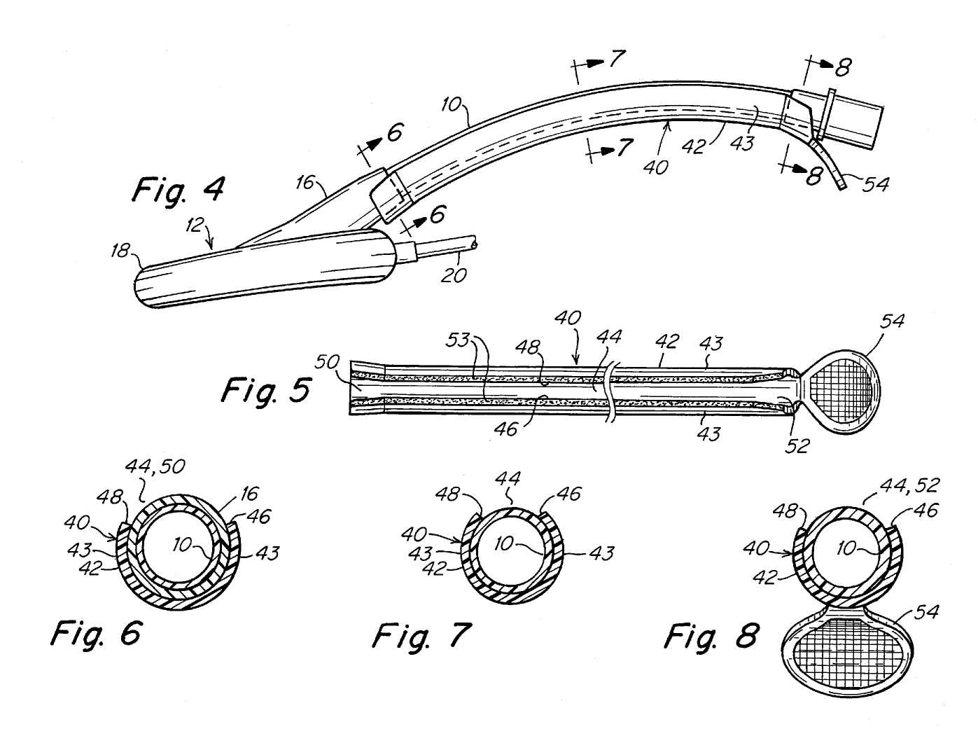

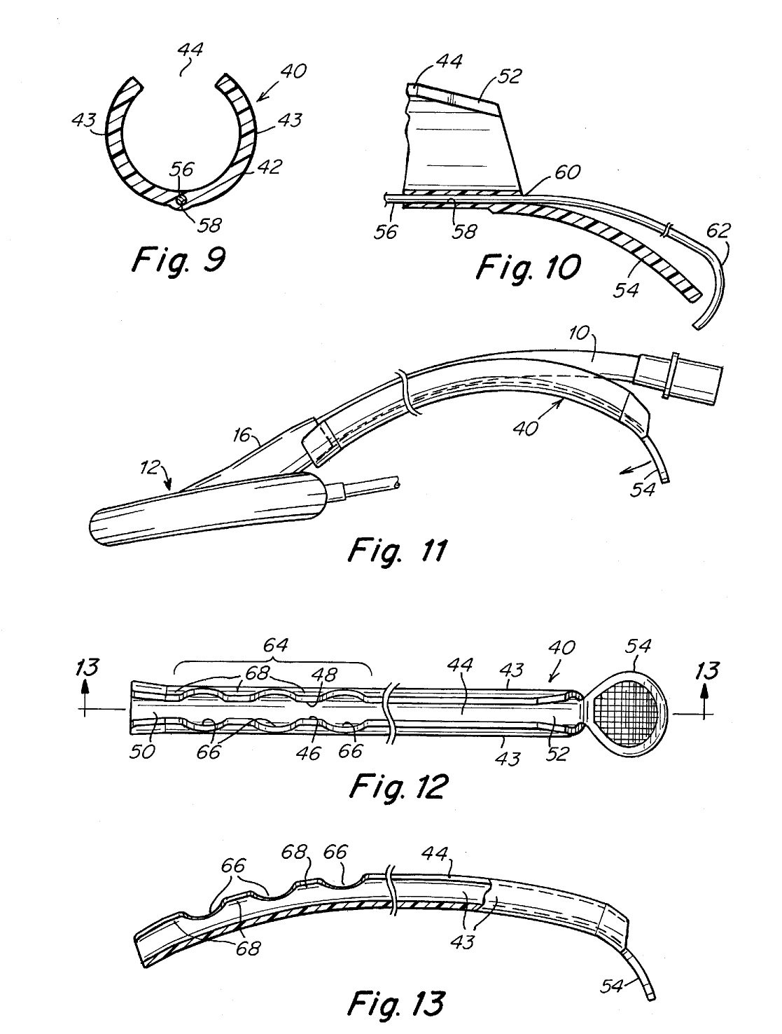

FIG. 4 illustrates, diagrammatically, a flexible LMA in combination with one embodiment of the brace 40 of the present invention to form an airway tube assembly arranged to be selectively configurable between flexible and stiffened states. The embodiment of the brace, shown in more detail in FIGS. 5-8, is illustrated in the form of a generally tubular member 42 having sides 43 and a longitudinally extending slot 44 formed posteriorly along a sagittal plane. The brace may be formed from a semi-rigid polymeric material such as PVC. The materials, structure and dimensions of the brace should be selected so that when mounted on the airway tube of the LMA the combination of the airway tube and brace is sufficiently rigid to enhance the ability of the device to be manipulated during placement. The brace is attached to the airway tube of the flexible LMA by spreading the posterior slit 44 and slipping the brace over the anterior side of the airway tube. The materials and dimensions of the brace should be selected with reference to the LMA with which it is to be used and should be sufficiently resilient so that the sides 23 of the brace will grip the airway tube. Preferably, the combined longitudinal stiffness of the airway tube 10 and brace 40 will be at least as great as the airway tube of a similarly sized “standard” LMA device and may be stiffer. After the braced LMA has been positioned and the cuff 18 inflated, the brace may be detached simply by peeling it off of the airway tube by holding the distal end of the airway tube and drawing the distal end of the brace toward the chin. The flexible airway tube then may be repositioned and secured in an out-of-the way location that does not interfere with the subsequent procedures. The brace should be formed from materials that will provide sufficient friction with the material of the airway tube of the LMA to prevent the brace from slipping. I have found that a brace formed from PVC works well with commercially available LMAs. The materials and dimensions may be varied to provide the desired degree of temporary stiffness to the airway tube assembly as will by appreciated by those skilled in the art.

As shown in FIGS. 5 and 6 the edges 46, 48 of the brace that define the longitudinal slot 44 are shaped to widen the proximal and the distal ends of the slot 44, the proximal end 50 of the slot being widened to facilitate placement of the proximal end of the brace over the airway tube 10 and the fitting 16 at which the airway tube is connected to the mask. The proximal end of the airway tube 10 also may be flared to accommodate the shape of the fitting 16. The distal end 52 of the slot 44 is widened to ease initial separation of the brace by peeling it away from the airway tube. The distal end of the brace also may be provided with a tab 54 that is easily grasped to initiate peeling off of the brace as suggested in FIG. 11. Once the peeling of the brace has been started at the distal end, the more proximal portions of the brace 40 separate easily from the airway tube 10. Should it become necessary to reposition the mask, for example, if the seal of the cuff to the laryngeal inlet becomes broken, the LMA can be removed, the brace can be replaced quickly and easily on the airway tube and the device can be reinserted and positioned in the patient. The widened ends 50, 52 of the slot 44 facilitate re-mounting the brace on the LMA. Preferably, the edges of the brace that define the slot 44 are marked, as by lines 53 that highlight their location. Most LMAs have a sagittal line that can be used as a reference in aligning the slot 44.

Because of variations in the oropharyngeal anatomies of patients it may be desirable, in some cases, to form the airway tube of the LMA to have a curve adapted for a particular patient. To that end the brace 40 may be provided with a manually shapeable stiffener 56 extending longitudinally along the brace. The shapeable stiffener 56 may be in the form of a wire-like stylet formed from a malleable material, (e.g., a metal such as aluminum or steel) that will retain its shape when bent. The stiffener 56 may be incorporated into the brace within a lumen 58 extending longitudinally of the brace. The lumen 58 may be formed directly in the wall of the brace or may be formed as a separate tube attached to the exterior of the brace. The stiffener and its lumen are located along the anterior of the brace and extend along a sagittal plane, opposite the posterior slot 44. The shapeable stiffener may be embedded permanently into the brace or may be detachable, for example by providing an opening 60 at the distal end of the lumen 58 (FIG. 10). The distal end of the detachable stiffener may have an easily grasped handle 62. The material and dimensions of the stiffener should be selected so that the combined airway tube and brace will retain the curve to which the device has been manually shaped.

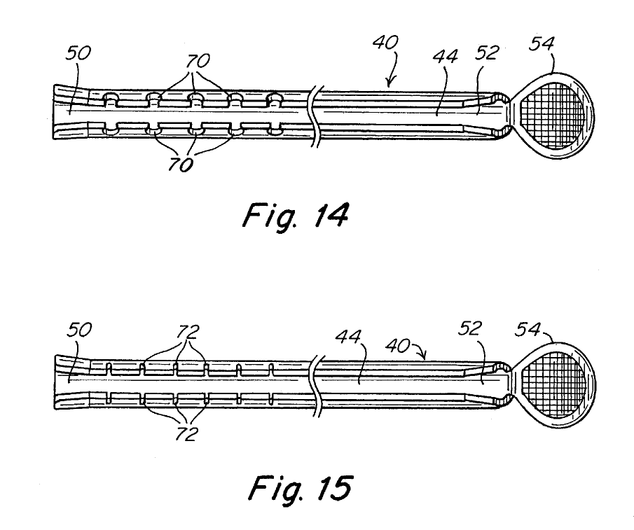

FIGS. 12-15 illustrate variations in the brace configuration in which the more proximal portions of the brace are more flexible than the distal portion in order to enable the proximal portion to bend more easily to conform to a patient's anatomy. Typically the sharpest portion of the curve of the patient's orolaryngeal anatomy will be in the region that will be occupied by the proximal portion of the LMA. By fabricating that portion of the brace to present less resistance to longitudinal bending the resulting force of the LMA during installation will be less likely to cause trauma. To that end, the proximal segment 64 of the brace 40 may be modified by forming cutout segments 66 at spaced locations along the edges that define the slot 44. FIGS. 12 and 13 illustrate such an arrangement in which the cut-outs may be in the form of longitudinally spaced scallops, with the remaining sidewall portions 68 of the brace sufficiently circumscribing the airway tube 10 to grip the airway tube 10 and maintain its bracing function to facilitate to control during positioning of the mask. FIG. 14 illustrates another arrangement for reducing the longitudinal stiffness of the proximal portion of the brace in which a plurality of smaller teardrop shaped cutouts 70 may be formed. FIG. 15 shows yet another alternative in which slits 72 are formed at longitudinally spaced intervals along the proximal portion of the brace.

Thus, the invention provides an arrangement and method by which the ability to place a laryngeal mask airway having a relatively flexible airway tube is enhanced. By temporarily stiffening the airway the LMA may be manipulated with at least the same facility as standard LMAs while also reducing the risk of trauma to oropharyngeal tissues and structures.

It should be understood, however, that the foregoing description of the invention is intended merely to be illustrative and that other embodiments, modifications and equivalents may be apparent to those skilled in the art without departing from the scope of the invention and the accompanying claims.

I claim:

1. A laryngeal mask assembly comprising:
a laryngeal mask airway having a flexible airway tube with proximal and distal ends and a laryngeal mask at the proximal end of the airway tube, the airway tube being sufficiently flexible so that when the mask is placed at the laryngeal inlet, the portion of the airway tube that extends out of the patient's mouth can be flexed to and secured in an out-of-the way position;
a brace detachably mounted on the airway tube, the brace being constructed to grip the airway tube and increase the stiffness of the combined airway tube and brace sufficiently to enable the mask to be positioned at the laryngeal inlet by manipulation of the braced airway tube;
whereby after the mask has been positioned at the laryngeal inlet the brace may be detached from the airway tube and the portion of the airway tube that extends out of the patient's mouth can be secured in an out-of-the-way position.

2. A laryngeal mask airway device comprising:
an airway tube assembly comprising an airway tube having proximal and distal ends and a laryngeal mask at the proximal end;
the airway tube assembly being selectively configurable between flexible and stiffened states, the stiffened state enabling the position of the mask to be adjusted about a patient's laryngeal inlet by manipulation of the airway tube assembly and the flexible state enabling the airway tube to be secured in an out-of-the-way position without affecting the position of the mask.

3. A laryngeal mask airway device as claimed in claim 2 wherein the airway tube assembly comprises a brace detachably mountable on the airway tube, the combined stiffness of the airway tube and brace being greater than that of the airway tube alone.

4. A laryngeal mask airway device as claimed in claim 3 wherein the brace comprises:
a generally tubular member having anterior and posterior regions and a slot extending posteriorly along the length of the tubular member and along a sagittal plane, the sides of the brace being flexible and resilient to enable the airway tube to be removably inserted transversely into and gripped by the tubular member.

5. A laryngeal mask airway device as claimed in claim 4 wherein the edges that define the slot are marked to visually identify those edges.

6. A laryngeal mask airway device as claimed in claim 4 wherein the mask has a fitting at which the airway tube is attached and wherein the proximal end of the brace is flared to engage the fitting.

7. A laryngeal mask airway device as claimed in claim 4 wherein the slot is widened at its proximal end.

8. A laryngeal mask airway device as claimed in claim 4 wherein the slot is widened at its distal end.

9. A laryngeal mask airway device as claimed in claim 4 wherein the slot is widened at its proximal and distal ends.

10. A laryngeal mask airway device as claimed in claim 4 further comprising:
the brace having a manually shapeable stiffener extending along its length and adapted to be bent to impart a selected curve to the brace, the stiffener being constructed to retain the braced airway tube in the selected curve.

11. A laryngeal mask airway device as claimed in claim 10 wherein the manually shapeable stiffener is removable from the brace.

12. A laryngeal mask airway device as claimed in claim 10 wherein the manually shapeable stiffener is embedded in the brace.

13. A laryngeal mask airway device as claimed in claim 4 wherein the manually shapeable stiffener is formed from a malleable metal.

14. A laryngeal mask airway device as claimed in claim 4 further comprising a manually graspable tab connected to the distal end of the anterior side of the brace.

15. A laryngeal mask airway device as claimed in claim 3 wherein a proximal segment of the brace is more flexible than the more distal portion of the brace.

16. A laryngeal mask airway device as claimed in claim 15 wherein the more flexible proximal segment is defined by a plurality of cutout segment formed in the sidewalls of the brace.

17. A method for ventilating or supplying anesthesia to a patient's lungs comprising:
providing a laryngeal mask airway having a flexible airway tube with proximal and distal ends and a laryngeal mask at the proximal end;
attaching a stiffening brace to the airway tube to form an assembly having a combined stiffness greater than that of the airway tube alone;
inserting the braced laryngeal mask airway into the patient's oropharynx and positioning the mask at the laryngeal inlet by manipulating the braced airway tube; and
with the mask in position, separating the brace form the airway tube.

18. A method as claimed in claim 16 further comprising,
thereafter securing the airway tube in an out-of-the-way position without affecting the position of the mask.
